(19) United States
(12) Patent Application Publication
(10) Pub. No.: US 2024/0407778 A1
(43) Pub. Date: Dec. 12, 2024

(54) FULLY-THREADED BIOABSORBABLE SUTURE ANCHOR

(71) Applicant: Arthrex, Inc., Naples, FL (US)

(72) Inventors: Peter J. Dreyfuss, Naples, FL (US); Stephen S. Burkhart, Boerne, TX (US)

(21) Appl. No.: 18/808,716

(22) Filed: Aug. 19, 2024

Related U.S. Application Data
(63) Continuation of application No. 17/739,806, filed on May 9, 2022, now Pat. No. 12,064,104.
Application No. 17/739,806 is a continuation of application No. 16/784,612, filed on Feb. 7, 2020, now Pat. No. 11,324,493.
Application No. 16/784,612 is a continuation of application No. 15/382,855, filed on Dec. 19, 2016, now Pat. No. 10,595,847.
Application No. 15/382,855 is a continuation of application No. 11/224,060, filed on Sep. 13, 2005, now Pat. No. 9,521,999.

(51) Int. Cl. A61B 17/04; A61B 17/00; A61L 17/10; A61L 17/14

(52) U.S. Cl. CPC A61B 17/0401; A61L 17/10; A61L 17/145; A61B 2017/00004; A61B 2017/0409; A61B 2017/0414; A61B 2017/044; A61B 2017/0445; A61B 2017/0458

(57) ABSTRACT

A suture anchor includes an anchor body having a longitudinal axis, a proximal end, and a distal end. The anchor body includes a central bore located at the proximal end and a passage extending between the central bore and the distal end. The central bore has a first dimension substantially perpendicular to the longitudinal axis, and the passage has a second dimension substantially perpendicular to the longitudinal axis less than the first dimension. The suture anchor includes a flexible strand extending through the passage, and a first knot formed in the flexible strand at the distal end has a third dimension greater than the second dimension to prevent the first knot from passing through the passage. A closed loop is positionable at least partially within the central bore and formed entirely by the flexible strand without forming any additional knots on the flexible strand extending proximally from the first knot.

### REFERENCE TO RELATED APPLICATION

This application is a continuation of U.S. patent application Ser. No. 17/739,806 filed on May 9, 2022, now U.S. Pat. No. 12,064,104 granted Aug. 20, 2024; which is a continuation of U.S. patent application Ser. No. 16/784,612 filed on Feb. 7, 2020, now U.S. Pat. No. 11,324,493 granted May 10, 2022; which is a continuation of U.S. patent application Ser. No. 15/382,855 filed on Dec. 19, 2016, now U.S. Pat. No. 10,595,847 granted Mar. 24, 2020; which is a continuation of U.S. patent application Ser. No. 11/224,060 filed on Sep. 13, 2005, now U.S. Pat. No. 9,521,999 granted Dec. 20, 2016.

### FIELD

The present invention relates to an apparatus and method for anchoring surgical suture to bone. More specifically, the present invention relates to arthroscopic apparatus and methods for anchoring suture to bone using a fully-threaded bioabsorbable suture anchor having a loop inserted into the suture anchor.

### BACKGROUND

When soft tissue tears away from bone, reattachment becomes necessary. Various devices, including sutures alone, screws, staples, wedges, and plugs have been used in the prior art to secure soft tissue to bone. Recently, various types of threaded suture anchors have been developed for this purpose. Some threaded suture anchors are designed to be inserted into a pre-drilled hole. Other suture anchors are self-tapping.

Problems can arise if the structure for attaching the suture fails, allowing the suture to become detached from the anchor. Also, the suture often is exposed to abrasion or cutting by sharp or rough areas along the walls of the bone canal into which the anchor is inserted.

Further, the prior art suture anchors having eyelets extending from the proximal ends require countersinking of the eyelet below the bone surface to avoid having the patient's tissue abrade against the exposed eyelet. As a result, suture attached to the eyelet is vulnerable to abrasion by the bony rim of the countersunk hole into which the suture anchor is installed. In addition, in biodegradable prior art devices, the eyelet can degrade rapidly, causing the suture to become detached from the anchor prematurely.

Accordingly, a need exists for a threaded suture anchor to which suture is secured effectively so as to prevent detachment of the suture and eliminate anchor “pull back.” In addition, a need exists for suture anchors having eyelets that will not abrade tissue and do not require countersinking. Suture anchors having a small core diameter providing maximum pullout strength even in soft bone and maximum suture fixation strength are also needed.

### SUMMARY

In an embodiment, a suture anchor includes an anchor body having a longitudinal axis, a proximal end, and a distal end. The anchor body includes a central bore located at the proximal end and a passage extending between the central bore and the distal end. The central bore has a first dimension substantially perpendicular to the longitudinal axis, and the passage has a second dimension substantially perpendicular to the longitudinal axis less than the first dimension. The suture anchor includes a flexible strand extending through the passage, and a first knot formed in the flexible strand at the distal end has a third dimension greater than the second dimension to prevent the first knot from passing through the passage. A closed loop positionable at least partially within the central bore of the anchor body is configured to be formed entirely by the flexible strand without forming any additional knots on portions of the flexible strand extending proximally from the first knot.

In another embodiment according to any of the previous embodiments, the closed loop is positionable completely within the anchor body.

In another embodiment according to any of the previous embodiments, the closed loop is located within the central bore of the anchor body.

In another embodiment according to any of the previous embodiments, the central bore has an opening configured to receive a driver head for driving the suture anchor.

In another embodiment according to any of the previous embodiments, at least one suture strand is configured to pass slidingly through the flexible strand.

In another embodiment according to any of the previous embodiments, the anchor body is threaded from the proximal end to the distal end.

In another embodiment according to any of the previous embodiments, the anchor body comprises a bioabsorbable material.

In another embodiment according to any of the previous embodiments, the flexible strand includes a suture formed of ultrahigh molecular weight polyethylene.

In another embodiment according to any of the previous embodiments, the first knot is coated with a glue material.

In another embodiment according to any of the previous embodiments, a distal surface of the central bore is substantially perpendicular to the longitudinal axis of the suture anchor.

In another embodiment according to any of the previous embodiments, the anchor body is a single, integral, monolithic component.

In another exemplary embodiment, a suture anchor assembly for attachment of tissue to bone includes a suture anchor including an anchor body having a distal end, a proximal end, a longitudinal axis, an outer surface, a central bore located at the proximal end, and a passage extending between the central bore and the distal end. The central bore has a first dimension substantially perpendicular to the longitudinal axis, and the passage has a second dimension substantially perpendicular to the longitudinal axis that is less than the first dimension. The suture anchor assembly also includes a first flexible strand that extends through the passage of the anchor body, and a first knot formed in the first flexible strand at the distal end of the anchor body has a third dimension greater than the second dimension to prevent the first knot from passing through the passage. A closed loop positionable at least partially within the central bore of the anchor body is configured to be formed entirely by the first flexible strand without forming any additional knots on portions of the first flexible strand extending proximally from the first knot. The suture anchor assembly also includes at least a second flexible strand attached to the suture anchor and configured to pass slidingly through the first flexible strand.

In another embodiment according to any of the previous embodiments, the closed loop is configured to be recessed from the proximal end of the anchor body by about one third the length of the anchor body.

In another embodiment according to any of the previous embodiments, the anchor body comprises a plurality of thread flights extending from the outer surface of the anchor body.

In another embodiment according to any of the previous embodiments, the anchor body has an outside diameter of about 5.5 mm.

In another exemplary embodiment, a method attaches a suture anchor assembly to a driver. The suture anchor assembly is configured to approximate tissue to bone and includes a suture anchor. The suture anchor includes an anchor body having a distal end, a proximal end, a longitudinal axis, a central bore located at the proximal end, and a passage extending between the central bore and the distal end. The central bore has a first dimension substantially perpendicular to the longitudinal axis, and the passage has a second dimension substantially perpendicular to the longitudinal axis that is less than the first dimension. The suture anchor assembly includes a first flexible strand that extends through the passage of the anchor body, and a first knot formed in the first flexible strand at the distal end of the anchor body has a third dimension greater than the second dimension to prevent the first knot from passing through the passage. A closed loop positionable at least partially within the central bore of the anchor body is configured to be formed entirely by the first flexible strand without forming any additional knots on portions of the first flexible strand extending proximally from the first knot. The suture anchor assembly includes at least a second flexible strand attached to the suture anchor and configured to pass slidingly through the first flexible strand. The method includes the steps of coupling the suture anchor assembly to the driver and threading ends of the second flexible strand through a cannula of the driver.

In another embodiment according to any of the previous embodiments, the second flexible strand further comprises a needle attached to one end.

In another embodiment according to any of the previous embodiments, comprising the step of securing the needle within a cavity of a handle of the driver.

Other features and advantages of the present invention will become apparent from the following description of the invention, which refers to the accompanying drawings.

### BRIEF DESCRIPTION OF THE DRAWINGS

FIG. 1 is a cross-sectional view of a fully-threaded suture anchor according to the present invention, and with two suture strands looped through the internal loop of the fully-threaded suture anchor.

FIG. 2 is a perspective view of the fully-threaded suture anchor of FIG. 1, but without the two suture strands looped through the internal loop of the fully-threaded suture anchor shown in FIG. 1.

FIG. 3 is a proximal end view of the fully-threaded suture anchor of FIG. 2.

FIG. 4 is a cross-sectional view of the fully-threaded suture anchor of FIG. 2.

FIG. 5 is a distal end view of the fully-threaded suture anchor of FIG. 2.

FIG. 6 is another perspective view of the fully-threaded suture anchor of FIG. 2.

FIG. 7 (a) is a perspective view of a first exemplary embodiment of a cannulated driver preloaded with the fully-threaded suture anchor of FIGS. 1-6.

FIG. 7 (b) is an enlarged view of the fully-threaded suture anchor of FIG. 7 (a).

FIG. 8 (a) is a perspective view of a second exemplary embodiment of a driver preloaded with the fully-threaded suture anchor of the present invention, and with needles attached to the two suture strands looped through the internal loop of the fully-threaded suture anchor.

FIG. 8 (b) is an enlarged view of the needles attached to the fully-threaded suture anchor of FIG. 8 (a).

FIG. 9 (a) is a perspective view of a third exemplary embodiment of a driver preloaded with the fully-threaded suture anchor of the present invention, and with punch needles attached to the two suture strands looped through the internal loop of the fully-threaded suture anchor.

FIG. 9 (b) is an enlarged view of the fully-threaded suture anchor of FIG. 9 (a).

FIG. 10 is a perspective view of the cannulated driver shown in FIG. 7 (a).

FIG. 11 is another perspective view of the cannulated driver of FIG. 10.

FIG. 12 is a side view of the cannulated driver of FIG. 11.

FIG. 13 is a cross-sectional view of the handle of the cannulated driver of FIG. 10.

FIG. 14 is a side view of the handle of the cannulated driver of FIG. 10.

FIG. 15 is a top view of the handle of the cannulated driver of FIG. 10.

FIG. 16 is a top view of the driver shown in FIG. 8 (a).

FIG. 17 is a perspective view of the driver shown in FIG. 16.

FIG. 18 is a side view of the shaft and head of the driver shown in FIG. 16.

FIG. 19 is a top view of the shaft and head shown in FIG. 18.

FIG. 20 is a top view of the handle of the driver of FIG. 16, with the cover removed.

FIG. 21 is a proximal end view of the handle shown in FIG. 20.

FIG. 22 is a partial cross-sectional side view of the driver of FIG. 20.

FIG. 23 illustrates a schematic view of the fully-threaded suture anchor of FIG. 1, having a recessed loop, and with two suture strands looped through the internal loop of the fully-threaded suture anchor.

FIG. 24 illustrates a top view of the cannulated driver of FIGS. 10-15 loaded with the fully-threaded suture anchor of FIG. 23, and with the two suture strands threaded through the cannula of the driver.

FIG. 25 illustrates a top view of the cannulated driver of FIGS. 10-15 loaded with the fully-threaded suture anchor of FIG. 23, and showing the loading path of the two suture strands threaded through the cannula of the driver.

FIG. 26 illustrates a side view of the cannulated driver of FIGS. 10-15 loaded with the fully-threaded suture anchor of FIG. 23, and showing the two suture strands threaded through the cannula of the driver and secured to the driver.

FIG. 27 illustrates a side view of the driver shown in FIGS. 16-22 before being loaded with the fully-threaded suture anchor having two suture strands with attached needles.

FIG. 28 illustrates a schematic view of the fully-threaded suture anchor of FIG. 1, having a recessed suture loop and two suture strands looped through the loop of the fully-threaded suture anchor, the two suture strands having attached needles.

FIG. 29 illustrates a perspective view of the driver of FIG. 27 loaded with the fully-threaded suture anchor of FIG. 28, and with the two suture strands with attached needles partially threaded through a side cannulation of the driver.

FIG. 30 illustrates another perspective view of the driver of FIG. 27 loaded with the fully-threaded suture anchor of FIG. 28 and with the two suture strands and attached needles secured to the driver.

FIG. 31 is a perspective view of a punch employed to create a pilot hole for the fully-threaded suture anchor of the present invention.

FIG. 32 is a distal end view of the punch of FIG. 31.

FIG. 33 is a schematic cross-sectional view of a bone fragment undergoing a suture anchor installation in accordance with a method of the present invention and at an initial stage.

FIG. 34 illustrates the bone fragment of FIG. 33 at a stage of suture anchor installation subsequent to that shown in FIG. 33.

FIG. 35 illustrates the bone fragment of FIG. 33 at a stage of suture anchor installation subsequent to that shown in FIG. 34.

FIG. 36 illustrates the bone fragment of FIG. 33 at a stage of suture anchor installation subsequent to that shown in FIG. 35.

### DETAILED DESCRIPTION

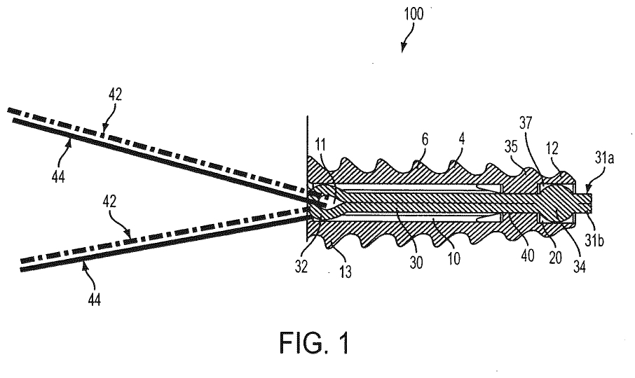

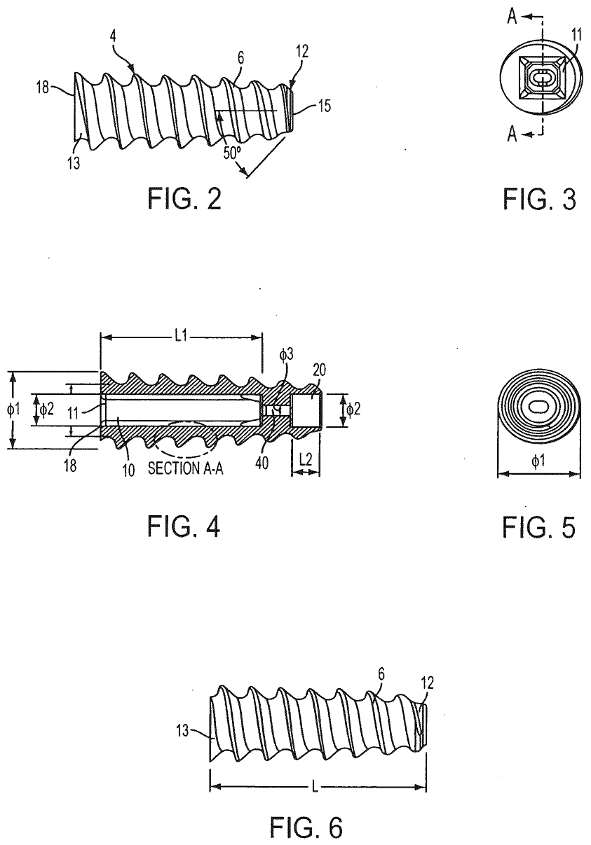

Referring now to the drawings, where like elements are designated by like reference numerals, FIGS. 1-6 illustrate a fully-threaded bioabsorbable suture anchor 100 of the present invention. The fully-threaded suture anchor 100 includes a body 4 provided in the shape of a tapered cylinder and having a distal end 12 and a proximal end 13.

As shown in FIG. 1, the fully-threaded suture anchor 100 is provided with a continuous thread 6 which wraps around the body 4 in a clockwise direction, the crest of the threads tapering from wide to narrow from the proximal to the distal end of the anchor. The proximal threads of anchor 100 with the widest crest surfaces are designed to engage the thin cortical shell in osteopenic bone to prevent anchor “pull back,” which could cause the back of the anchor to be proud to the bone. In an exemplary embodiment, suture anchor 100 is provided with about eight thread flights wrapping around body 4, with the angle of the proximal surface of each thread being approximately between one-third and one-fourth the angle of the distal surface of each thread relative to the horizontal. For example, in the preferred embodiment, the proximal surface of each thread has an angle of 12° relative to a plane horizontal to the axis of the suture anchor, while the distal surface of each thread has an angle of 45° relative to the same horizontal plane.

By providing the suture anchor 100 with continuous threads running from the tip to the proximal end where the driver engages, the thread potential is maximized and the wasted space is minimized. The fully-threaded design of the anchor of the present invention substantially improves pull-out strength compared to suture anchors with protruding eyelets, and prevents anchor “pull-back” that can occur with countersunk anchors.

As shown in FIGS. 1 and 4, the suture anchor 100 has a cylindrically shaped bore or socket 10 starting from a rectangularly shaped opening 11 at the proximal end and extending into the anchor body 4 approximately two-thirds of the length therethrough. Distally of the bore 10, anchor body is provided with a knot socket 20 which extends from the distal end 12 of the anchor. Knot socket 20 may have various shapes and configurations, for example, a cylindrical shape, as shown in FIG. 5. Bore 10 communicates with knot socket 20 through passage 40.

The proximal surface and associated edges of suture anchor 100 defining the rectangularly shaped opening 11 is rounded and smooth. Preferably, the proximal surface of the suture anchor 100 forming the periphery of the opening 11 forms a rounded lip 18 (FIG. 4) so that opening 11 has a slightly wider diameter than the main portion of the cylindrical bore 10. With the smooth and rounded proximal end provided in the anchor of the present invention, sutures threaded through the bore 10 and opening 11 will not be abraded by any sharp edges, and will not become frayed upon pressure or rubbing against the anchor at the proximal opening.

In a preferred embodiment, diameter Ø₂ (FIG. 4) of opening 11 is substantially equal to that of the knot socket 20, but larger than diameter Ø₃ of the passage 40 and smaller than outside diameter Ø₁ of the anchor body 4. Preferably, the outside diameter Ø₁ of the anchor body 4 is about 3 to about 8 mm, more preferably of about 5.5 mm, and the diameter Ø₂ of the opening 11 and socket 20 of about 1.5 to about 4.5 mm, more preferably of about 3 mm. In an exemplary embodiment, body 4 of suture anchor 100 has a length of about 0.6 in. and an exterior diameter of about 0.22 in. (5.5 mm) as measured across the outer diameter of the threading at the proximal end of the anchor.

Referring back to FIG. 1, a flexible strand 30, preferably suture, is threaded into the anchor body 4, with one end of the suture strand being threaded through the socket 20, passage 40 and bore 10 to form a loop or eyelet 32 located at least partially within bore 10 at the proximal end 13 of the anchor 100. Ends 31a, 31b extending through the anchor from the distal end 12 of the suture anchor are tied to form at least one knot 34.

In the preferred embodiment illustrated in FIG. 1, the loop 32 is completely recessed from the proximal end 13 of the anchor body 4. However, the invention also contemplates embodiments according to which the loop 32 extends out of the suture anchor 100 by a distance of about 0.5 to about 1.5 mm, more preferably of about 1 mm. In alternative configurations, the loop 32 may be also located completely outside of the bore 10 of the anchor 100. Thus, although the embodiment of FIG. 1 illustrates eyelet 32 disposed completely within the anchor body 4, this embodiment is only illustrative and the invention is not limited to it. The position and size of the eyelet of the present invention is determined according to the characteristics of the arthroscopic procedure, and the need to precisely orientate the eyelet during anchor insertion to optimize suture sliding characteristics.

The fully-recessed loop 32 of FIG. 1 has the ability to self-align, eliminating the need to determine eyelet alignment as it applies to the orientation of the tissue edge. The fully-recessed loop 32 also enhances suture slideability compared to conventional anchors with protruding eyelets, and allows the suture to slide against the smooth inner edge of the anchor reducing the potential for suture abrasion from the cortical bone edge.

At the distal end 12 of anchor 100, flexible strand of material 30 forms at least one knot 34, which is preferably an over-hand knot. Knot 34 is housed in the knot socket 20 and rests on most distal surfaces 37 of regions 35 of the anchor body 4 that define the passage 40 having a diameter narrower than that of the sockets 10 and 20. To increase the pull out strength of the strand 30 from the anchor, knot 34 may be optionally coated with a glue material to increase its strength and facilitate adherence to the walls of the socket 20. Knot 34 increases the pullout strength of the strand even in soft bone, provides increased fixation, and eliminates the anchor “pull back.”

The strand 30 may be formed of any flexible material. In the preferred embodiment, strand 30 and loop 32 are formed of a high strength suture material such as the one described in U.S. Pat. No. 6,716,234 to Grafton et al., the disclosure of which is incorporated by reference in its entirety. In additional embodiments, the strand 30 may be insert-molded into the anchor in the manner described in U.S. Pat. No. 5,964,783 to Grafton et al., the disclosure of which is also incorporated by reference in its entirety.

The anchor body 4 is preferably formed of a translucent or transparent polymer material, and is preferably made of bioabsorbable materials such as polyglycolic or polylactic acid polymers. Accordingly, flexible strand 30 is visible through the body of the fully-threaded anchor 100 to provide visual confirmation of strand encapsulation within the anchor. Advantageously, the flexible strand of material 30 and the anchor body 4 are made of materials selected such that the loop 32 will not biodegrade before anchor body 4. As used in the present application, the term “bioabsorbable” is considered to be interchangeable with the term “biodegradable,” “resorbable,” and “absorbable” to mean that the device can be absorbed by the body over time. Also, the measurements, angles and ratios between the dimensions of the suture anchor may be varied from those described above so as to be suitable for the conditions and applications in which the suture anchor is to be used.

Optionally, the suture anchor can be distributed with at least one strand of suture already threaded through the loop 32. For example, FIG. 1 illustrates suture strands 42, 44 attached to the loop allowing the sutures to slide smoothly with minimal friction. In an exemplary embodiment, the suture strands 42, 44 may be FiberWire composite sutures of alternating colors to maximize repair strength, aid in suture management and provide superior tying characteristics.

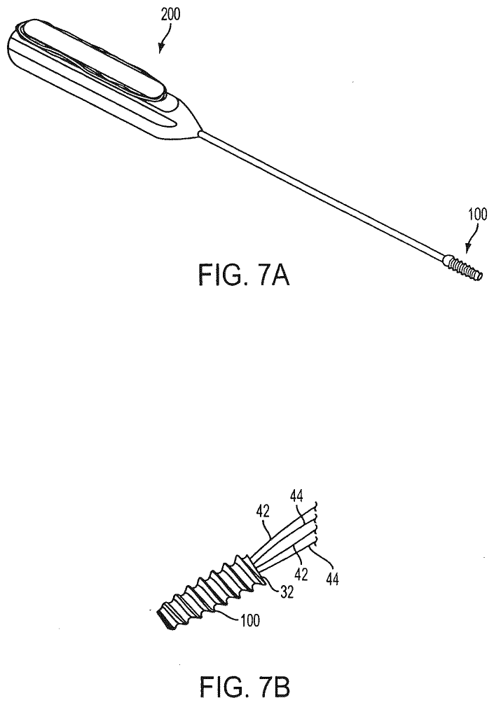

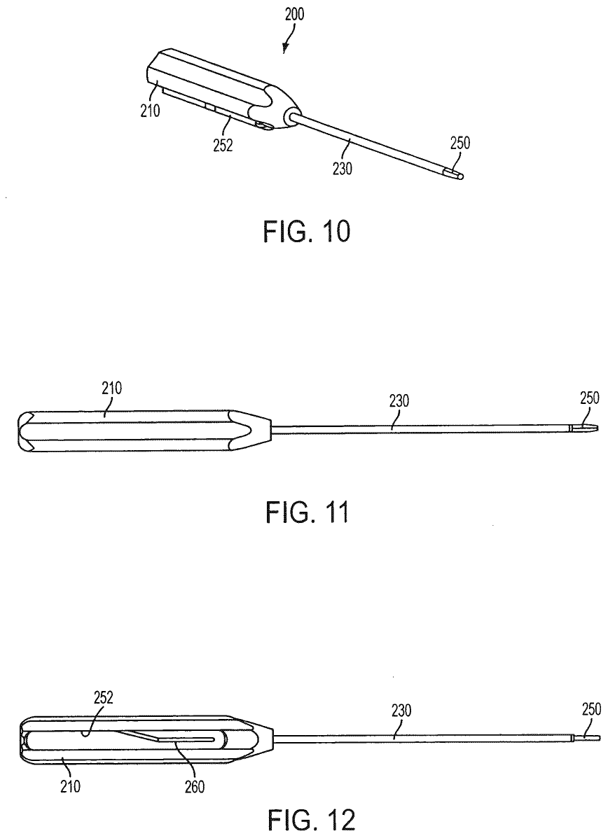

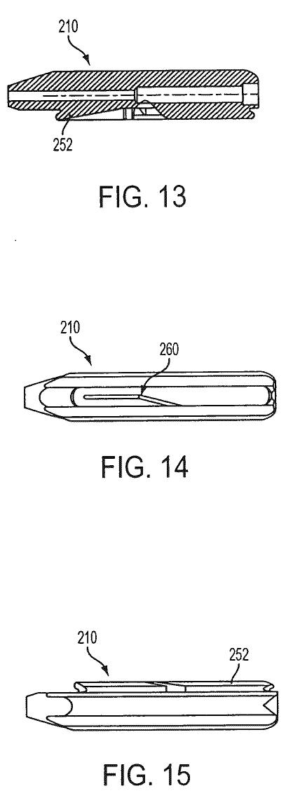

FIGS. 7 (a), 8 (a) and 9 (a) illustrate various embodiments of drivers 200, 300, 400 used to install the fully-threaded suture anchor 100 of the present invention. FIG. 7 (a) illustrates cannulated driver 200 preloaded with the fully-threaded suture anchor of FIGS. 1-6 and with suture strands 42, 44 attached to the loop 32. As explained in more detail below with reference to FIGS. 10-15 and 23-26, suture strands 42, 44 are threaded through the cannula of the driver 200 and secured on a hook on the handle of the driver, to allow the distal end of the head of the driver to be inserted into the opening 11 and bore 10 of the anchor 100 so that the suture anchor is driven into a pilot hole.

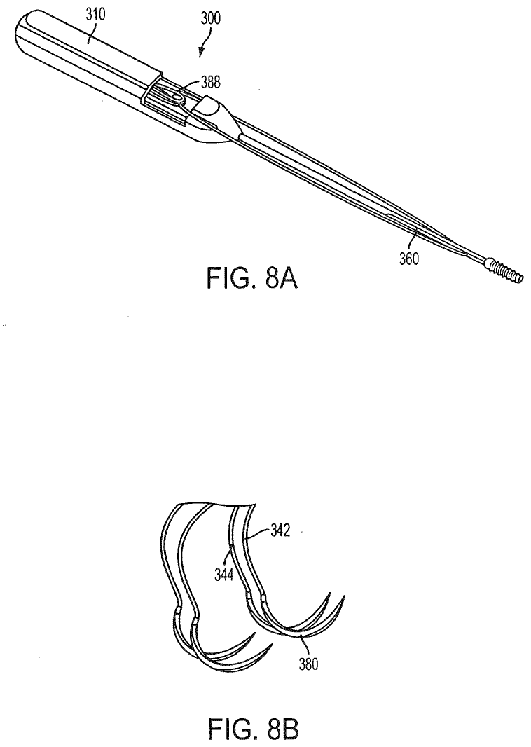

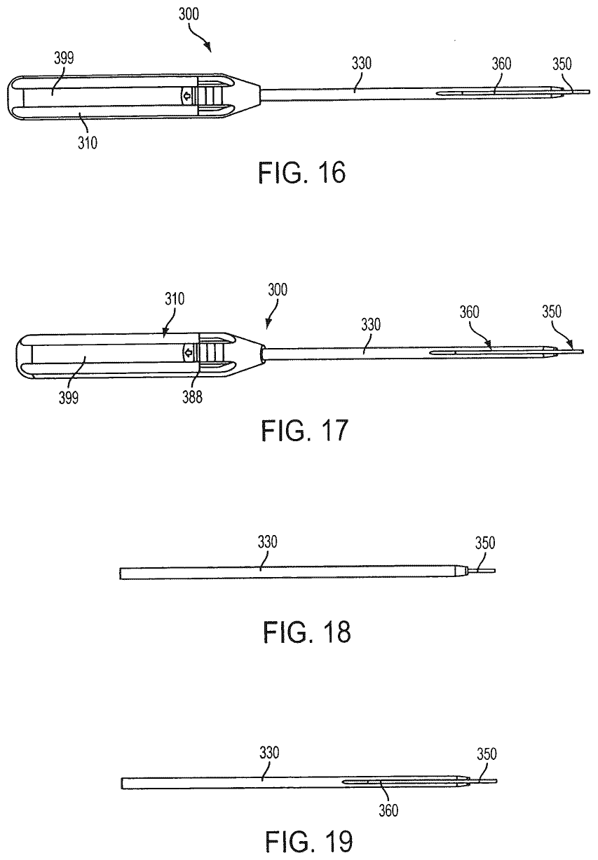

FIG. 8 (a) illustrates a second embodiment of a driver of the present invention. Driver 300 (which will be described in more detail in FIGS. 16-22) is not cannulated, but rather presented with a slot or side cannulation 360 in the shaft 330. The slot allows suture strands that are provided with needles 380 (FIG. 8 (b)) and that are attached to the loop 32 of the anchor 100 to pass through the slot and around the sides of handle 310, to be further secured in needle slot 388 of the handle.

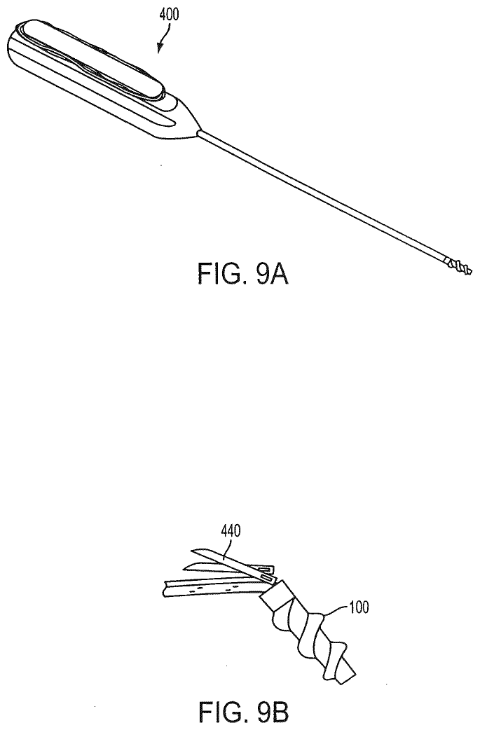

FIG. 9 (a) illustrates yet another embodiment of a driver of the present invention, according to which driver 400 is employed in connection with suture strands with punch needles 440 (FIG. 9 (b)) that are attached to the loop 32 of the anchor 100 of the present invention.

FIGS. 10-15 illustrate details of the first exemplary embodiment of driver 200 used to install the fully-threaded suture anchor 100 of the present invention during an arthroscopic procedure. The driver 200 is provided with a head 250, a shaft 230 and a handle assembly 210. The head 250 of the driver is configured to be received within anchor socket 10 of the fully-threaded suture anchor 100 of FIGS. 1-6. In an exemplary embodiment, the drive head is rectangularly shaped and has a width and a length which substantially corresponds to the width and length of opening 11 in suture anchor 100. Preferably, the drive head is slightly shorter and has a slightly smaller width than opening 11, so that the fit is not too tight, yet ensures secure engagement for driving the suture anchor into bone.

The shaft 230 preferably comprises an elongate, narrow diameter body suitable for use in remote procedures performed through percutaneous tissue punctures, such as arthroscopic, laparoscopic and other invasive procedures and the like. The shaft typically has a length of about 5 cm to about 20 cm, preferably about 15 cm. The diameter of the shaft assembly is sufficiently small to facilitate introduction through access sheaths, cannulas, trocars, and the like, typically being less than about 10 mm, preferably about 5 mm.

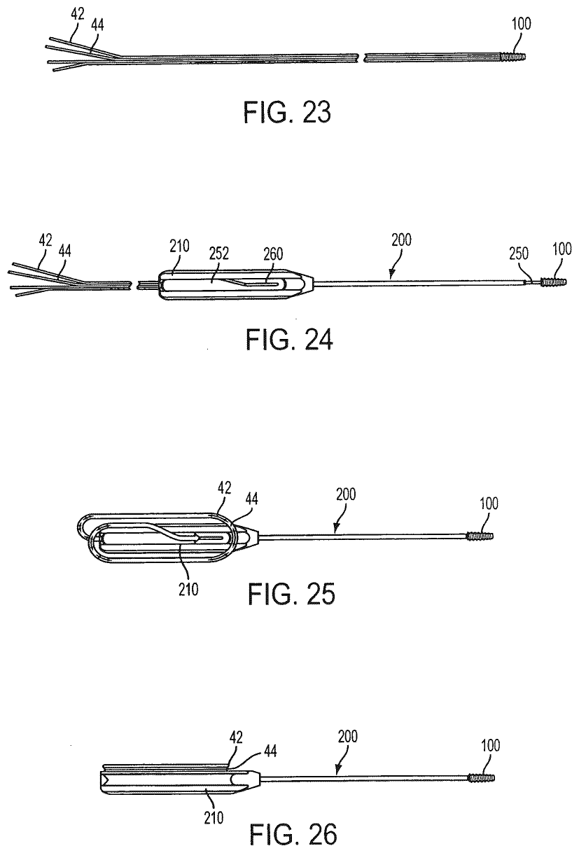

The handle assembly 210 preferably includes an elongated double hook 252 extending substantially along the length thereof and having a hook at the proximal end and at the distal end thereof, and a clip 260 formed at one end region of the double hook 252. As explained in more detail below with reference to FIGS. 23-26, when driver 200 is engaged with suture anchor 100, excess lengths of suture 42, 44 passed through the proximal end of driver 200 can be wrapped around the double hook 252, and the ends of the sutures can be secured in the clip 260. In this manner, the suture strands 42, 44 can be prevented from becoming tangled or otherwise interfering with the surgeon's work.

Driver 200 is preferably constructed to withstand an application of about 20 in/lb of torque. Preferably, although not necessarily, at least the shaft and drive head are made of stainless steel. However, other materials may be used which provide the necessary strength and rigidity for installing the suture anchor of the present invention into cortical bone.

The anchor 100 and driver 200 may be provided to the surgeon as a preformed assembly with the sutures 32, 34 pre-threaded through loop 32 and through the cannula of the driver and secured on the handle.

FIGS. 16-22 illustrate details of the second exemplary embodiment of driver 300 used to install the fully-threaded suture anchor 100 of the present invention during an arthroscopic procedure, preferably during an open procedure such as mini-open rotator cuff repairs. The driver 300 is different from the driver 200 described above in that driver 300 allows installation of a fully-threaded suture anchor that has attached at least one strand of suture with a surgical needle. Thus, driver 300 is not cannulated but rather comprises a slot or side cannulation 360 provided for about half the length of the shaft 330 and defined by break edges 350. The side cannulation allows suture strands 342, 344 (FIG. 8 (b)), which are received in the cannulation, to be provided with surgical needles 380 (FIG. 8 (b)) at one end, which would not be possible if the driver had a central (fully closed) cannulation.

When driver 300 is engaged with suture anchor 100, excess lengths of suture 342, 344 with attached needles 380 can be secured in recess region or cavity 388 (FIG. 8 (b)) of the handle 310. The cavity 388 is accessed by opening a pivotable hatch 399. In this manner, the needles 380 and suture strands 342, 344 are wrapped around tie-down bars disposed in the inside of the housing cavity when the hath is closed. Thus, surgical needles 380 may be safely stored within the handle 310, preventing therefore any piercing of surgical gloves and any problems in maintaining the needles sterile.

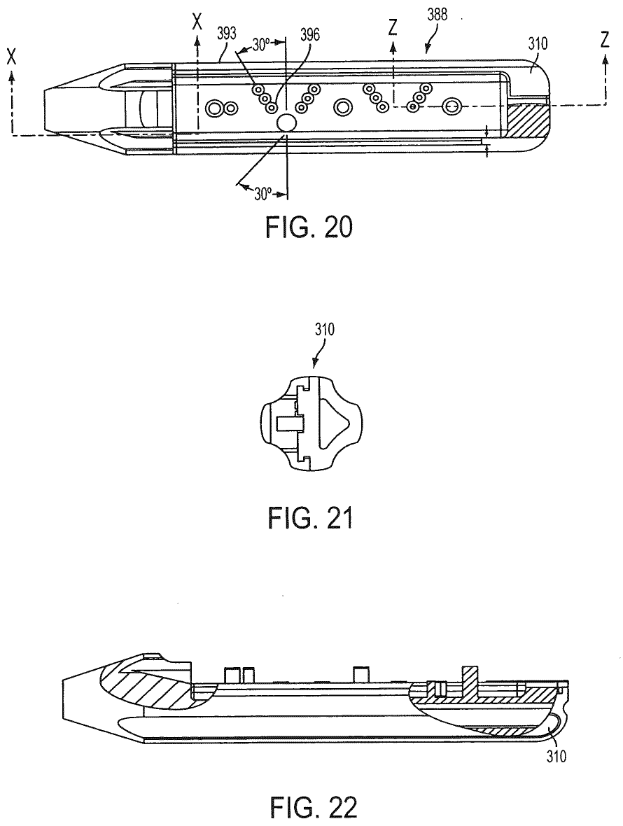

FIGS. 20-23 illustrate details of the housing cavity 388 provided in the handle 310 of the driver 300. As shown in FIG. 20, cavity 388 is provided with a plurality of slots 396 and tie-down bars 393, that allow the surgical needles 380 to be “parked” or secured within the slots and the bars. If desired, a plurality of sutures with or without needles may be housed within the housing cavity 388. The cavity 388 is accessed by opening the pivotable hatch 399. Driver 300 is also configured to be received within anchor opening 11 of the fully-threaded suture anchor 100 of FIGS. 1-6.

FIGS. 23-26 illustrate the cannulated driver of FIGS. 10-15 loaded with the fully-threaded suture anchor of FIG. 23. As shown in FIG. 24, the two suture strands 42, 44 are first threaded through the cannula of the driver 200, and the distal end of the drive head 250 (FIG. 24) of the driver is then inserted into the opening 11 of the anchor 100. The sutures exiting the proximal end of driver 200 are wrapped around the double hook 252 and/or clipped in clip 260.

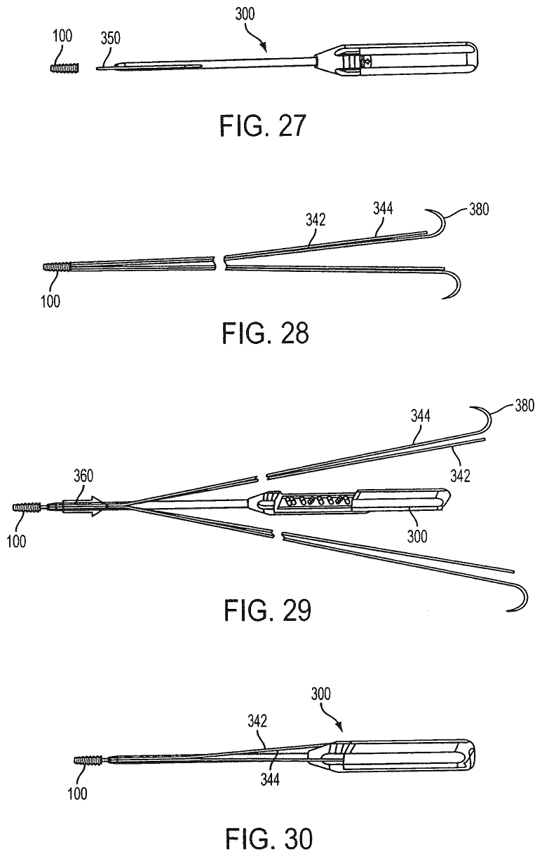

FIGS. 27-30 illustrate the driver 300 of FIGS. 16-22 loaded with the fully-threaded suture anchor 100 and provided with two suture strands 342, 344 having attached surgical needles 380. As shown in FIG. 29, the two suture strands 342, 344 are first passed through side cannulation 360 of the shaft of the driver 300, so that the distal end of the drive head 350 of the driver is then inserted into the opening 11 of the anchor 100. The sutures passed through the side cannulation are pulled toward the proximal end of the driver 300 and the suture strands 342, 344 are wrapped around the tic-down bars 393 in the housing cavity 388, while the needles 380 are stored within slots 396.

Sutures anchors according to the present invention can be used for arthroscopic procedures. The anchors are also advantageous for open and mini-open surgical procedures. Specific examples of applicable procedures include cortical bone-soft tissue fixation, Bankart and SLAP shoulder repairs.

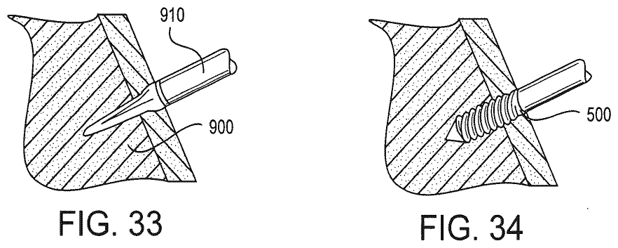

An exemplary method of employing the suture anchors of the present invention is described below with reference to FIGS. 33-36. FIG. 33 illustrates a schematic cross-sectional view of bone segment 900 which undergoes suture anchor installation according to the present invention. A punch 910 (FIG. 33) either alone or in combination with tap 500 (FIG. 34) may be employed for forming a hole in bone 900 into which suture anchor 100 is to be inserted. If bone 900 is soft bone, punch 910 may be sufficient for drilling the hole. If, however, the bone is hard cortical bone, the punch/tap combination may be desirable. Preferably, the diameter of the hole formed is slightly (e.g. 1 mm) smaller than the diameter of the suture anchor to be installed, to ensure good purchase of the suture anchor threads in the bone. Alternatively, a self-drilling/self-tapping suture anchor can be formed and inserted directly into bone by engaging the anchor with a driver and turning the anchor to advance the anchor directly into bone without previous formation of a hole.

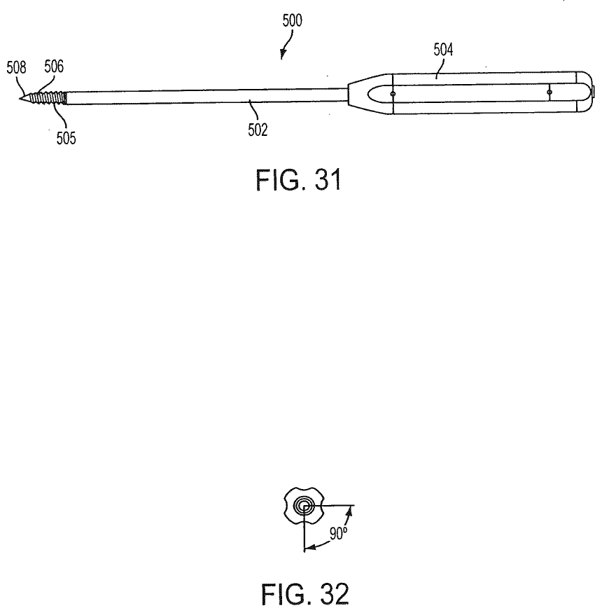

FIGS. 31 and 32 illustrate an embodiment of tap 500 employed to prepare a bone socket or pilot hole prior to insertion of the anchor. As shown in FIGS. 31 and 32, tap 500 includes a shaft 502 having a handle 504 on a proximal end and a tapping head 506 on a distal end. Tapping head 506 includes a trocar tip 508 followed by a tapered, spiral cutting section 505. Using tap 500, a hole for the suture anchor is formed to accommodate a head of a cannulated driver used to install the suture anchor.

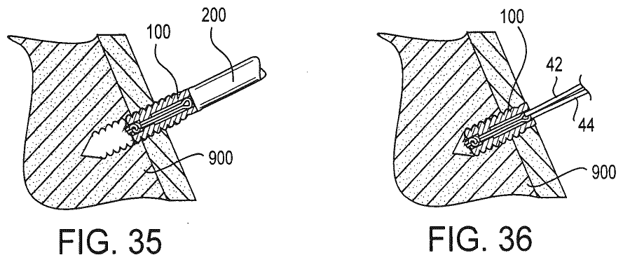

Reference is now made to FIGS. 35 and 36. Driver 200 loaded with the fully-threaded suture anchor of the present invention (shown in FIG. 26, for example) is then placed at the opening of the prepared hole in bone 900, and the driver 200 is rotated until the proximal surface of the anchor 100 is flush with the surface of the bone. The driver tip is then pulled back, to reveal the suture strands 42, 44 (FIG. 36). Since it is not necessary for the proximal end of the anchor to be countersunk below the bone surface to prevent tissue abrasion, the inventive anchor does not need to be inserted as far as prior art devices, and avoids abrasion of the sutures by the rim of the bone around the installed suture anchor.

The suture anchor of the present invention provides advantages in addition to those already discussed above. For example, with the threads provided along the entire length of the suture anchor body, the anchor is afforded maximum securement by the threads in the cortical bone, unlike some prior art anchors in which the threads only contact the cancellous bone. Also, by providing a knot which is optionally coated with a glue material, the suture anchor is installed with a higher torque than many prior art anchors, and thus has improved fixation strength.

Although the present invention has been described in relation to particular embodiments thereof, many other variations and modifications and other uses will become apparent to those skilled in the art.

What is claimed is:

1. A suture anchor comprising:
an anchor body having a longitudinal axis, a proximal end, and a distal end, wherein the anchor body includes a coaxial central bore located at the proximal end and a coaxial central passage extending between the central bore and the distal end, wherein the central bore has a first dimension substantially perpendicular to the longitudinal axis, and the central passage has a second dimension substantially perpendicular to the longitudinal axis that is less than the first dimension; and
a loop disposed at least partially within the anchor body, the loop having ends tied together in a knot disposed at the distal end of the anchor body.

2. The suture anchor of claim 1, wherein the loop is disposed completely within the anchor body.

3. The suture anchor of claim 1, wherein the knot is located within a socket disposed at the distal end of the anchor body.

4. The suture anchor of claim 3, wherein the loop is located within a bore of the anchor body.

5. The suture anchor of claim 4, wherein the bore has an opening configured to receive a driver head for driving the anchor.

6. The suture anchor of claim 1, further comprising at least one suture strand threaded through the loop.

7. The suture anchor of claim 6, wherein the suture strand further includes a needle attached at one end.

8. The suture anchor of claim 1, wherein the anchor body is threaded from the proximal end to the distal end.

9. The suture anchor of claim 1, wherein the anchor body comprises bioabsorbable material.

10. The suture anchor of claim 1, wherein the loop comprises a suture formed of ultrahigh molecular weight polyethylene.

11. The suture anchor of claim 1, wherein the knot is coated with a glue material.
